(19) United States
(12) Patent Application Publication
(10) Pub. No.: US 2006/0129076 A1
(43) Pub. Date: Jun. 15, 2006

(54) Broken collar bone fixing band

(76) Inventor: Naohiro Haneda, Toride-shi (JP)

Correspondence Address: RABIN & Berdo, PC, 1101 14TH STREET, NW, SUITE 500, WASHINGTON, DC, 20005, US

(21) Appl. No.: 10/534,717

(22) PCT Filed: May 20, 2003
(86) PCT No.: PCT/JP03/06278

§ 371 Date: Nov. 15, 2005

(30) Foreign Application Priority Data
Nov. 12, 2002 (JP) 2002-328524

(51) Int. Cl. A61F 5/00

(52) U.S. Cl. 602/19; 602/5

(57) ABSTRACT

A fractured clavicle fixing band comprises a back rest 1 rendered in contact with a back of a user along a backbone when used, a pair of shoulder belts 2 connected an end portion of the back rest, stretched as passed respectively from shoulders to axillae when used, and a chest front belt 3 for pulling and fastening, in front of a chest of the user, the pair of shoulder belts in a direction to narrow a distance therebetween, in which the front chest belt 3 is made of a nonflexible material. An orderly reset region of the user can be supported by the antagonistic action of a fastening force with the shoulder belt 2 and a fastening force with a chest belt 3.

### BACKGROUND OF THE INVENTION

1. Field of the Invention

This invention relates to a fractured clavicle fixing band used when fixing an orderly reset region after reset treatment of a fractured clavicle.

2. Description of Related Art

For treatment of any fractured bone of an entire body, it is necessary to support a region after subjected to reset treatment. Particularly where a clavicle is broken or fractured, such a treatment is given in which a fractured region is left until healed spontaneously with an orderly reset region fixed after reset by means of, e.g., manipulative reduction or the like. On fixing the orderly reset region at the time of the clavicle fracture, it is especially important to render a condition maintainable at all times, in which a patient throws out his chest sufficiently not to bend forward his both shoulders, since where unsupported shoulders are sagged forward, bone fragments respectively intrudes interiorly (long-axis shortening), and center fragments (at a side of a sternum) are dislocated in a posterosuperior direction upon pulled by a clavicular branch of a sternocleidomastoid muscle, and further peripheral fragments (at a side of a shoulder) are dislocated in an anteroinferior direction due to weight of an upper extremity. Upon maintaining the condition described above, such dislocation of the orderly reset region as forcing the bone fragments to swing at all times can be prevented, so that the fractured region can be healed in a short time without causing, e.g., such malnion as deforming the clavicle in a stepped shape.

To fix the orderly reset region at the time of the clavicle fracture as described above, such treatment had been conventionally provided, that a bandage, for example, is applied in a cross-coupled manner, however, since this treatment is complicated, a fractured clavicle fixing band has been invented to obtain more easily the same effect as that of the above treatment, and a fractured clavicle fixing band of this type is widely used in these days.

As a fractured clavicle fixing band of this type, for example, Japanese Patent Laid-Open Publication No. JA-H8-206,146 (hereinafter referred to as document 1) discloses a device (a clavicle fixing device) comprising a back rest portion formed in a belt shape, a pair of shoulder belts formed as connected in a letter Y shape to an upper end of the back rest portion, and an attachment method for coupling free ends of the shoulder belts to lower ends of the back rest portion in a detachably attachable manner as well as in a length-adjustable manner. The clavicle fixing device as disclosed in the patent document 1 has a structure allowing the clavicle fixing device to fit an anterior body-shape of an user upon absorbing individual differences by arranging the back rest portion along the backbone of the patient as well as by equipping a pad applied to an affected region, in a position-adjustable manner to each of the shoulder belts at the time that the shoulder belts are wore from the back portion through the clavicle region to the underarms.

Furthermore, Japanese Patent Laid-Open Publication No. JA-H9-299,391 (hereinafter referred to as document 2) discloses a band including a back rest extending downward along a spine, a waist belt coupled to a lower end of the back rest through a traction band made of an elastic material, a back belt formed as connected in an obliquely upward direction to an upper portion of the back rest, and a fixing belt coupled to a lower portion of the back rest, extending through the axilla to the shoulder position, having end portions with shoulder pads. This fractured clavicle fixing band as disclosed in the patent document 2 has a structure in which a ring for passing the back belt therethrough is formed on a turn-up projecting portion of the belt, sewn as attached to a center portion of the shoulder pad while a hook and loop fastener banded each other when turned down as overlapped is formed to each end portion of the back belt so that hook and loop fastener can be united where the back belt is turned down after passed through the ring formed on the shoulder pad, whereby the back rest can be pulled downward with appropriate force and a position of clavicle in an arcuate shape can be further reinforced. Furthermore, such a condition that the fixing belt bites into the axilla can be prevented, and such a condition that the shoulder pads fixes as pressing the affected region at all times can be maintained.

Furthermore, Examined Utility Model Publication No. JA-H6-23,285 (hereinafter referred to as document 3) discloses a band comprising a back rest having an elastic support rod, a shoulder belt extending from an upper portion of the back rest to the shoulder position, and an axillary belt extending from a lower portion of the back rest through the axilla to the shoulder position. This fractured clavicle fixing band as disclosed in the patent 3 can apply appropriate pressing force regardless of fractured regions or a body types since a shoulder pad is fixed to an end portion opposite the back rest, of either the shoulder belt or the axilla belt, and the shoulder pad and an end portion opposite the back rest, of the other belt are formed in a detachably attachable manner so a position as well as a mounting angle of the shoulder pad as to be adjustable and thus the shoulder pad can be freely applied to the most appropriate position of the fractured region.

On all of the above described conventional fractured clavicle fixing bands described in the document 1, the document 2, and the document 3, the belt (hereinafter explained upon generally referred to as a shoulder belt although differently named according to each of the documents) stretched as passed from the back through the upper portion of the shoulder and the axilla to the back again, are sufficiently fastened and the patient maintain a condition rendering his chest thrown out only with the above described redress in a backward direction so the orderly reset region to be fixed and supported as needed.

However, on fixing the orderly reset region at the time of the clavicle fracture, the redress in the backward direction in using the shoulder belt, as described above, is not enough as the fixing force. In particular, where the shoulder belt as described above is strongly fastened with force of a hundred percent when supporting the orderly reset region at the time of the clavicle fracture, a groove occurs between left and right shoulder blades (a central region of the spine) on the back, thereby causing a pain at the cowl muscle or the supraspinatus muscle, or other than that, causing excessive tension at the muscle in the neck, the shoulder, the back, or the like to result in stiffness in the shoulder, a pain in the joint, etc. Furthermore, the nerves or arteries in the axilla are pressed to possibly cause the neural paralysis. It is therefore undesirable to fasten the shoulder belt with force of that much, i.e., a hundred percent, so that in reality, each patient adjusts the force to fasten the shoulder belt within the appropriate range from approximate seventy to ninety percent. With fastening force within the above range, the swing in backward and forward directions of shoulder cannot be effectively prevented, so that the orderly reset region becomes excessively difficult to be fixed as supported for supporting purpose. The force within the above range is therefore not enough as the fixing force, as described above.

Where the orderly reset region is supported with the insufficient fixing force, a period up to healing, which is estimated to approximately four weeks under the condition that the orderly reset region is fixed sufficiently, is undesirably prolonged to six weeks or longer, thereby forcing physical load to the patient in vain. Furthermore, not only such a physical load, but also the mental stress coming from anxiety about possible prolongation of healing or financial pressure due to increased medical expenses is imposed on the patient. Moreover, where the orderly reset region is not sufficiently supported, not only is the period up to healing prolonged but also the malnion such as, e.g., the deformation in a stepped shape, refracture, or the like is easily caused, and such cases of a great number are actually found.

Furthermore, a device such as disclosed in Japanese Patent Laid Open Publication No. JA-2001-522290 (hereinafter referred to as document 4) has been provided as the conventional fractured clavicle fixing band, other than that of the above described type in which the orderly reset region is to be supported only with the redress in the backward direction in using the shoulder belt. The structure disclosed in the document 4 does not have the shoulder belt likewise each above described embodiment, but has a structure in which both of left and right thoracic panels respectively having sleeves redress the orderly reset position in the backward direction by a front transversely extending means, a back transversely extending means, and a straps connected to both shoulder parts of the thoracic panels through a rear of the neck. This device disclosed in the document 4 is described as used for the purpose of, e.g., treatment of the clavicle, promotion of healing of the fracture or the deformed clavicle, or the like.

However, since the device described in the document 4 comprises the front transversely extending means including the elastic straps such as elastically deformed upon the backward and forward swing of the shoulder along with breathing, coughing, or sneezing, both of left and right thoracic panels are difficult to be fixed as supported firmly. Where the thoracic panels cannot be firmly fixed as supported, the backward and forward swing of the shoulder cannot be suppressed sufficiently, and the orderly reset region thus becomes excessively difficult to be fixed as supported for supporting purpose at the time of the treatment of the clavicle. As a result, there is a risk of causing the same problems as in a case of the shoulder belt described above.

### DISCLOSURE OF THE INVENTION

This invention has been invented against the above described back ground, and aims to provide a fractured clavicle fixing band enable an orderly reset region to be fixed sufficiently without fastening such as leading to excessive tension at the muscle or pressure on nerves or arteries in the axilla.

The fractured clavicle fixing band according to this invention to achieve the above described purpose comprises a back rest rendered in contact with the back of an user along the backbone when wore, a pair of shoulder belts connected to one end portion of the back rest, stretched as passed from the shoulders through the axillae when wore, and chest front belt for pulling and fastening, in front of the chest of the user, the pair of shoulder belts in a direction to narrow a distance therebetween. The fractured clavicle fixing band according to this invention is characterized in that the chest front belt is formed of a non-expandable material.

The fractured clavicle fixing band thus structured according to this invention has the chest front belt formed of a non-expandable material, fastening in left and right directions in a manner to narrow the distance between the shoulder belts generating fastening force forcing the user to throw his chest out. According to this invention, the antagonistic action occurs between fastening force with the shoulder belts and fastening force with the chest front belt, thereby causing more stable fixing force. Thus, according to this invention, the action with the chest front belt provides stronger stability to such an effect as pulling the bone fragments posterosuperior, caused upon the function of the shoulder, such as redressing the clavicle in a backward direction and pressuring the clavicle in directions of external ends thereof, so that the clavicle region completely stops swinging in backward and forward directions, thereby preventing effectively the orderly reset region from being dislocated. According to this invention, since the orderly reset region is supported effectively as described above, side effects can be largely suppressed, such as, e.g., pain or stiffness in the shoulder due to excessive tension at the muscle in the neck, the shoulder, the back, etc., or the neural paralysis at the axilla or such as leading to the pressure of the axillary artery due to pain in the joint or the pressure of the axillary nerve, conventionally caused upon fastening the shoulder belt excessively strongly.

On the fractured clavicle fixing band according to this invention, the back rest may be equipped with a back-rest pad having one main surface in contact with the user's back set to a curved surface, having a prescribed height enough to render the user threw his chest out. According to this invention as described above, the orderly reset region can be suppressed more effectively from being dislocated upon enabling the user even in a supine position to keep his chest thrown out sufficiently, so that significant positional change of the body in backward and forward directions between, e.g., the daytime and nighttime, which has conventionally led to the problem such as displacement of the bone fragments, can be solved.

### BRIEF DESCRIPTION OF THE DRAWINGS

FIG. 1 is a view showing a structure of a fractured clavicle fixing band according to the first embodiment;

FIG. 2 is a side view showing a state where a back-rest pad is equipped to a back rest of the fractured clavicle fixing band;

FIG. 3 is a view illustrating a state where an user wears the fractured clavicle fixing band as well as showing a state where the user is viewed from a front side;

FIG. 4 is a view illustrating action of the back-rest pad in a state where the user is in a supine position;

FIG. 5 is a view illustrating a state where the user wears the fractured clavicle fixing band having a chest front belt of different structure as well as showing a state where the user is viewed from a front side;

FIG. 6 is a view illustrating a state where the user wears the fractured clavicle fixing band having a chest front belt of other different structure as well as showing a state where the user is viewed from a front side;

FIG. 7 is a view illustrating a state where the user wears a fractured clavicle fixing band according to the second embodiment as well as showing a state where the user is viewed from a front side;

FIG. 8 is a view illustrating a state where the user wears the fractured clavicle fixing band as well as showing a state where the user is viewed from a rear side;

FIG. 9 is a front elevation view showing a state where the back-rest pad is equipped to the back rest of the fractured clavicle fixing band; and

FIG. 10 is a side view showing a state where the back-rest pad is equipped to the back rest of the fractured clavicle fixing band.

### BEST MODE FOR CARRYING OUT THE INVENTION

Hereinafter, specific embodiments with application of this invention will be described in detail in reference to drawings.

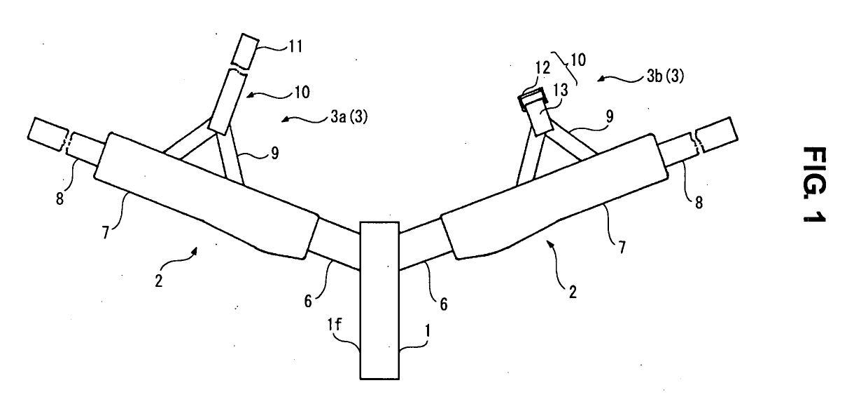

First, FIG. 1 shows a structure of the fractured clavicle fixing band according to the first embodiment with application of this invention. The fractured clavicle fixing band according to the first embodiment has a back rest 1, a shoulder belt 2, and a chest front belt 3.

The back rest 1 is a portion placed along the backbone of a patient wearing the fractured clavicle fixing bond (hereinafter referred to as an user), as in contact with the back. The back rest 1 is formed of, e.g., a fabric material having a pleasant texture and has a contact surface 1f in contact with the user's back, formed in a rectangular shape as well as rendered to touch softly the user. The back rest 1 is, to provide an appropriate rigidity, equipped with, e.g., a plate member of hard plastic, or a rigidity providing member (omitted to be illustrated) such as a plate of metal, e.g., stainless-steel. This back rest 1 is formed to have a length, on the user's back, approximately from a position slightly below the neck to a position substantially collinear with or slightly below the axilla.

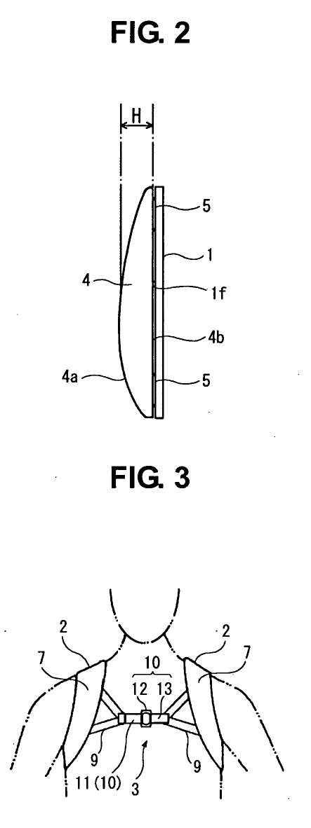

On the back-rest 1, as shown in FIG. 2, a back-rest pad 4 is detachably attached to a side of the contact surface 1f. This back-rest pad 4 has one main surface 4a as a side in contact with the user's back, in which a curved surface at a waist portion is set to a steeper slope than that at a neck portion so that a substantially center portion of the back-rest pad 4 has appropriate height H, and the main surface 4a is made of a material having appropriate elasticity, such as, e.g., a synthetic resin foam, a pressure relieving material, or the like, such as, e.g., styrofoam, urethane foam, or the like. On the back-rest pad 4, the surface portion in touch with the user's back is desirably formed of the pressure relieving material while a core member inside the surface portion is desirably made of the synthetic resin foam. On the back-rest pad 4, furthermore, the other main surface 4b as a side facing the contact surface 1f of the back rest 1 is set to a flat surface.

The back-rest pad 4 thus structured is, as described above, detachably attached to the back rest 1, in which a hook and loop fastener 5 as shown in FIG. 2, for example, is used as an attaching and detaching method to the back-rest 1. The hook and loop fastener 5 is in a flat shape as well as formed to the other main surface 4b facing the contact surface 1f of the back rest 1. Upon using the above described hook and loop fastener 5 as the attaching and detaching method, the back-rest pad 4 can be attached to and detached from the back rest 1 with a single touch, thereby being easily handled.

The back-rest pad 4 has, as described hereafter, a function similar to a back pillow such as rendering the user in a supine position, wearing the fractured clavicle fixing band according to this embodiment to threw his chest out upon pushing his back up so the orderly reset region as to be supported even when the user is in a supine position. Therefore, height H of the back-rest pad 4, which differs depending on the user's body type, is appropriately set within the range of, for one example, from three to five centimeters, and in practice, the back-rest pads 4 of several sizes within the above range are preferably prepared to be selectively used depending on the user's body type. The back-rest pad 4 can be selectively used with ease since allowed to be detached from and attached to the back-rest 1 with a single touch with the hook and loop fastener 5.

Appropriately, the back-rest pad 4 has the same or shorter width with respect to the backrest 1.

It is to be noted that the back rest 1 is not limited in a rectangular shape but may be in other shapes, e.g., a square shape or the like, while the back-rest pad 4 equipped to the back rest 1 is preferably in a shape such as fitting along a hollow appearing in the back of the user in a supine position with his back equipped with the back rest 1, and where limited in such shapes as described above, the back-rest pad 4 is not limited only in the above shape cited above as one structural example.

The pair of shoulder belts 2, i.e., two shoulder belts 2 defined as one set, are equipped to near one end portion in a longitudinal direction of the back rest 1 having a rectangular shape as described above, in particular, one end portion located at the neck portion in a case where the user wears the fractured clavicle fixing band, in a manner to render the fractured clavicle fixing band be symmetrical in a substantially letter Y shape as a whole. Each of the shoulder belts 2 comprises a coupling belt portion 6, a pad portion 7, and a fastening belt portion 8, all of which are formed as in a single belt form in totality. The coupling belt portion 6 is a portion for coupling the back rest 1 to the bad portion 7. The pad portion 7 is a portion stretched as passed from an upper portion of the shoulder through the axilla of the user at the time that the user wears the fractured clavicle fixing band, as described hereinafter. The fastening belt portion 8 is a portion engaged with the back rest 1 at the time that the pad portion 7 is stretched as passed as described above so the shoulder belt 2 as to be in a loop shape. Of these portions respectively composing the shoulder belt 2, the coupling belt portion 6 and the fastening belt portion 8 are formed of an ordinary fabric belt material while the pad portion 7 is formed with use of, e.g., a fabric material to have an appropriate cushioned property since stretched as passed from the shoulder through the axilla of the user as directly in contact with the user to fasten the user with force of a certain as described later. Furthermore, the pad portion 7 is formed to have a length enough to be stretched as passed from the shoulder to the axilla of the user as described above.

The chest front belt 3 is composed of two chest front belt halves 3a, 3b formed respectively to the pair of shoulder belts 2, in which each of the chest front belt halves 3a, 3b comprises a coupling belt portion 9 and a pulling and fastening belt portion 10 connected thereto. The coupling belt portion 9 is a portion for coupling the shoulder belt 2 to the pulling and fastening belt 10 upon connected to the pad portion 7 of the shoulder belt 2 at two connecting points as well as upon connected to the pulling and fastening belt portion 10 at one connecting point. The pulling and fastening belt portion 10 is a portion for coupling the chest front halves 3a, 3b to each other as well as for pulling and fastening a distance between the shoulder belts 2 at the time that the user wears the fractured clavicle fixing band as described later. The pulling and fastening belt portion 10 at the side of the chest front belt half 3a connected to one side of the shoulder belts 2, i.e., the shoulder belt 2 at a left side in FIG. 1 in this embodiment, is composed of a pulling and fastening belt 11 having one surface formed with a hook and loop fastener (omitted to be illustrated) while the pulling and fastening belt portion 10 at the side of the chest front belt half 3b connected to the other side of the shoulder belts 2, i.e., the shoulder belt 2 at a right side in FIG. 1 in this embodiment, is composed of a fastened belt 13 equipped with a buckle 12 in a square shape.

On the chest front belt 3, the pulling and fastening belt 11 and the fastened belt 13 are formed of an inflexible material, e.g., an ordinary fabric belt material, thereby being able to fasten as well as support a distance between the shoulder belts 2 with stable force when the chest front belt halves 3a, 3b are coupled to each other.

In this embodiment, the chest front belt halves 3a, 3b are respectively connected to the shoulder belts 2 in a manner to form a triangular shape with the shoulder belt 2 and the coupling belt portion 9 connected thereto 2 at two connecting points. This is because to exert fastening force with the chest front belt 3 toward a distance between the shoulder belts 2, in obliquely left and right directions, i.e., a cross-coupled manner, in front of the user's chest. It is to be noted that the connection between the chest front belt halves 3a, 3b and the shoulder belts 2 is not limited to in a manner of a triangular shape, but each of the front chest belt halves 3a, 3b may be connected to the shoulder belt 2 in using the coupling belt portion 9 formed in semicircular shape instead of the above triangular shape as long as the fastening force is to be exerted in a cross-couple manner in front of the user's chest.

The above described structure of the shoulder belt 2 is one example, and this invention is not limited thereto. For example, the shoulder belts may not be in the same shape each other such as a symmetrical shape with respect to the back rest 1 but may have one pad portion 7 stretched as passed onto the fractured region, formed larger than the other pad portion 7. Furthermore, the chest front belt halves 3a, 3b may be structured to be coupled in using the hook and loop fastener formed to each of facing surfaces, not to be coupled in the above described manner, i.e., coupled in using the buckle 12 in a square shape, equipped to one side of the chest front belt halves 3a, 3b, as described later.

A means for wearing the thus structured fractured clavicle fixing band is described. FIG. 3 shows a state where the user wears the fractured clavicle fixing band. It is to be noted that FIG. 3 is a view showing the user from a front side. In wearing the fractured clavicle fixing band, first, where the back-rest pad 4 is equipped to either the contact surface 1f of the back rest 1 or the back rest 1, one main surface 4a of the back-rest pad 4, set to a curved surface, is rendered to face the side of the user's back portion, and the connecting portions of the back rest 1 to the shoulder belts 2 is placed to the side of the neck while the rest back 1 is rendered, at the portion slightly below the neck, in contact with the back in a manner to be along the backbone of the user. Next, the shoulder belts 2 are respectively passed from the upper side to the front side of the shoulder, and furthermore stretched as passed through the axilla to the back. The fastening belt portion 8 is engaged with the back rest 1 with the engaging method formed to the other end portion of the back rest 1 at the waist portion, such as, e.g., the same buckle (omitted to be illustrated) that equipped to the fastened belt 13. The fastening belt portion 8 is engaged with the back rest 1 upon passed through the buckle at the back rest 1 and folded back with the buckle as the axis and thereafter fastened with, e.g., the hook and loop fastener formed to the fastening belt portion 8. On the fractured clavicle fixing band, at the time of engaging the fastening belt portion 8, the user is provided with the appropriate fastening force by the shoulder belts 2 upon pulling the fastening belt portion 8 toward the back portion.

Subsequently, on the fractured clavicle fixing band, the chest front belt 3 pulls and fastens a distance between the shoulder belts 2. The distance between the shoulder belts 2 is pulled and fastened upon coupling the chest front belt halves 3a, 3b connected respectively to the shoulder belts. The chest front belt halves 3a, 3b are coupled, in the same manner to engage the fastening belt portion 8 with the rest back 1 as described above, upon passing the pulling and fastening belt 11 at the side of the chest front bet half 3a through the buckle 12 of the pulling and fastening belt portion 10 at the side of the chest front belt half 3b as well as upon folding back the pulling and fastening belt 11 with the buckle 12 as the axis and then fastening the pulling and fastening belt 11 with, e.g., the hook and loop fastener formed thereto. On the fractured clavicle fixing band, at the time of coupling the chest front belt halves 3a, 3b, the user is provided with the fastening force by the chest front 3 in appropriate left and right directions, i.e., in a direction to narrow the distance between the shoulder bands 2, upon further pulling the fold back pulling and fastening belt 11.

On the fractured clavicle fixing band as worn in the above described manner, the shoulder belts 2 generating the fastening force forcing the user to be in a state where his chest is thrown out, is fastened with the chest front belt 3 in left and right directions in a manner to narrow the distance between the shoulder belts 2. Furthermore, on the fractured clavicle fixing band, the antagonistic action of the fastening force with the shoulder belts 2 and the fastening force with the inflexible chest front belt 3 thus occurs, thereby being able to generate the more stable fixing force. As a result, the action with the chest front belt provides 3 stronger stability to such an effect as pulling the bone fragments posterosuperior, caused upon the function of shoulder belts 2 such as redressing the clavicle in a backward direction and pressuring the clavicle in directions of external ends thereof, so that the clavicle region completely stops swinging in backward and forward directions including swinging at the time of breathing, and accordingly the orderly reset region can be prevented effectively from being dislocated.

On the fractured clavicle fixing band according to this invention, the fixing force such as enabling to support the orderly reset region of the fractured region is not exerted upon strengthening the fastening force with the shoulder belts 2, but the strong fixing force is exerted upon the antagonistic action caused upon forming the chest front belt 3 as described above. In this case, the chest front belt 3 pulls and fastens the shoulder belts 2 without causing cases such as excessive tension at the muscle or such as pressing nerves or arteries in the axilla, or even where any case is caused, there are extremely few cases. Therefore, the side effects can be largely suppressed, such as, e.g., pain or stiffness in the shoulder due to excessive tension at the muscle in the neck, the shoulder, the back, etc., or the neural paralysis at the axilla or the pressure of the axillary artery due to a pain in the joint or the pressure of the axillary nerve, conventionally caused when the shoulder belt 2 is fastened excessively strongly.

It has been conventionally common to loosen the shoulder belts 2 upon modifying to some extent the fastening of the shoulder belts 2 to prevent the above side effects, and therefore the fixing force becomes insufficient, thereby easily causing the orderly reset region to be dislocated due to the swing of the clavicle region. However, the fractured clavicle fixing band according to this invention can exert the strong fixing force because of the antagonistic action generated upon formation of the chest front belt 3, so that the orderly reset region can be supported firmly with preventing the occurrence of the above side effects.

Since the orderly reset region can be supported firmly, as for healing of the fractured clavicle, such cases can be prevented as, e.g., healing of the deformation such as in a stepped shape due to the insufficient support of the orderly reset region, the prolongation of the healing (delayed union), refracuture, or need for another operation caused as the worst case for the reason that the fractured bone are not healed but causes the pseudarthrosis upon the closure of the fractured edges due to the swing of the fractured fragment. As a result, the physical load, mental stress, and financial pressure due to the increase in the medical expenses, can be reduced.

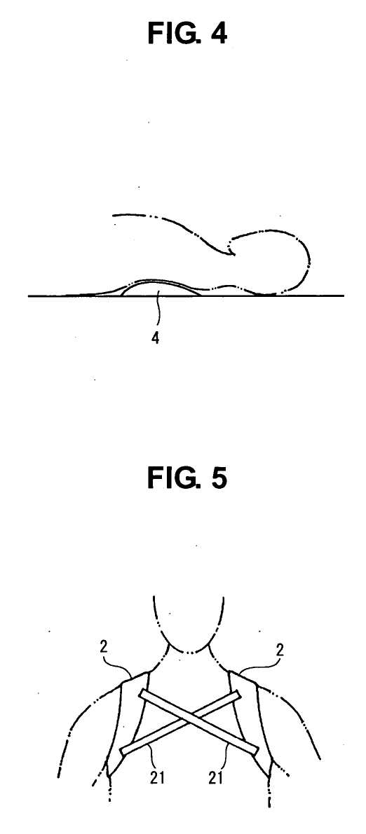

The fractured clavicle fixing band having the above described shoulder belts 2 and the chest front belt 3 for generating the fastening force causing the antagonistic action with respect to the fastening force with the shoulder belts 2 has effectively an sufficient effect for fixing and supporting the orderly reset region in a state where the user is in a sitting or standing up position. Upon equipment of the above described back-rest pad 4 to the back rest 1, the fractured clavicle fixing band according to this invention can fix and support the orderly reset region sufficiently even where the user is in a supine position, e.g., sleeping during bedtime. In particular, on the fractured clavicle fixing band, as shown in FIG. 4, the back-rest pad 4 having appropriate height H functions in a similar way to the back pillow such as pushing up the back of the user in a supine position so that the user even in a spine position can keep throwing his chest out sufficiently, thereby being able to prevent effectively the orderly reset region from being dislocated. Furthermore, since having height H in three dimensions while being located at an intermediate position between the shoulder belts 2 pulling the shoulder toward the back portion, i.e., located as rendered along the backbone, the back-rest pad 4 has a function similar to a fulcrum of a lever with respect to the fastening in using the shoulder belts 2, thereby facilitating the shoulder belts 2 fastening effectively. The fractured clavicle fixing band thus can keep the user's chest thrown out even when the user is in a supine position, thereby being able to prevent effectively the orderly reset region from being dislocated. Therefore, significant positional changes of the body in backward and forward directions between the daytime and nighttime, which has conventionally led to the problem such as displacement of the bone fragments, can be solved.

It is to be noted that the back-rest pad 4 does not need to function in a similar to the back pillow such as described above in a case where the healing of the fractured region proceeds to a certain extent. Where the healing reaches such a stage, it is necessary to facilitate the rehabilitation to elevate and extend the upper extremities so the burden on body movement in daily life as to be reduced. Furthermore, depending on the body type of the user (e.g., overweight body type), there are cases where such a function similar to the back pillow becomes unnecessary even before the healing proceeds, with the purpose of reducing the burden on body movement in daily life. From this point of view also, the back-rest pad 4 is desirably attached in a detachable manner to the back rest 1 with the detaching and attaching method such as, e.g., the hook and loop fastener 5 as described above.

The above explanation describes that the chest front belt 3 formed to the fractured clavicle fixing band according to this embodiment is composed of a pair of the chest front belt halves 3a, 3b, but this invention is not limited to such a structure. As long as generating the puling and fastening force in left and right directions in a manner to narrow the distance between the pair of shoulder belts 2 stretched as passed from the upper position of the shoulder through the axilla, the chest front belt 3 according to this invention can be applicable with any structure, e.g., a structure in which, likewise a chest front belt 21 as shown in FIG. 5, two chest front belts 21 are formed as one set while being equipped to the shoulder belts 2 upon crossed in front of the chest. The chest front belts 21 as described above can also exert the fastening force in a obliquely direction in a cross-coupled manner, thereby being able to pull and fasten the distance between the shoulder belts 2 in left and right directions.

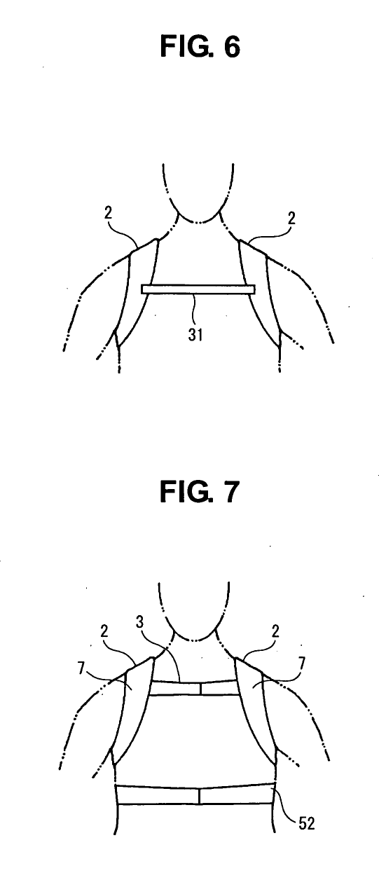

Furthermore, not likewise the chest front belts 3, 21, as described above, such as connected to a single shoulder belt 2 at two connecting points to exert the fastening force in a cross-coupled manner, such a chest front belt is also applicable, as exerting the fastening force in a horizontal direction to pull and fasten the distance between the shoulder belts 2 in left and right directions, likewise a chest front belt 31 as shown in FIG. 6. It is to be noted that FIG. 6 shows the fractured clavicle fixing band formed with a single chest front belt 31 but the number of the chest front belt 31 is not limited, so the chest front belts 31 of more than two numbers may be certainly applicable while the chest front belt 31 may be composed of two belt halves likewise the chest front belt 3.

The fractured clavicle fixing band according to the second embodiment in this invention will be explained next. It is to be noted that in this embodiment, the same numerals are assigned to elements having the same structure as the fractured clavicle fixing band according to the first embodiment to omit the detailed explanation.

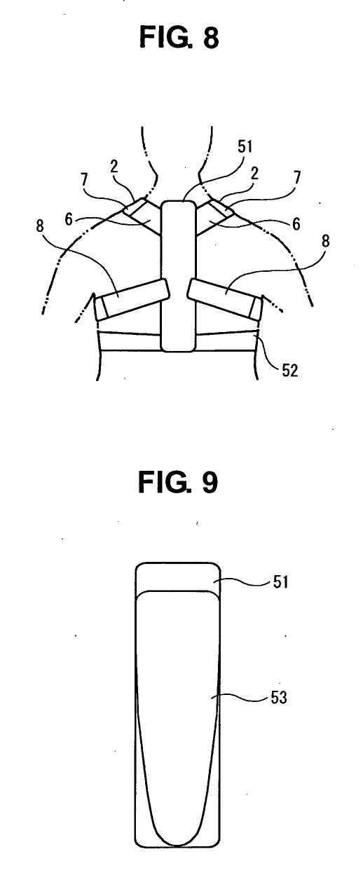

As shown in FIG. 7 and FIG. 8, the fractured clavicle fixing band according to the second embodiment has a back rest 51, the shoulder belts 2, the chest front belt 3, and a fixing belt 52. The back rest 51 of the fractured clavicle fixing band according to the second embodiment has a length longer than that of the back rest 1 of the fractured clavicle fixing band according to the first embodiment, to be more specific, the back rest 51 has a length approximately from a position slightly below the neck to an approximately center portion of the back of the user, compared with the back rest 1 having a length approximately from a position slightly below the neck to a position substantially collinear with or slightly below the axilla when the user wears the fractured clavicle fixing band.

It is to be noted even where the back rest 51 is rendered to have the above described length, to provide the user with sufficient fastening force by the shoulder belts 2, it is appropriate to render a position where the shoulder belts 2 are engaged with the back rest 51 is the same as that of the other end portion at the waist portion, of the back rest 1 of the fractured clavicle fixing band according to the first embodiment 1.

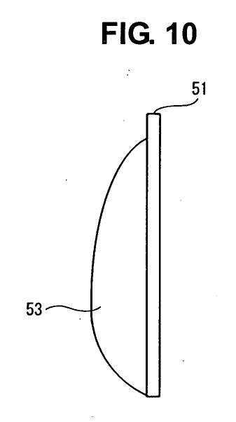

The back rest 51 is also equipped in detachably attachable manner with a back-rest pad 53. As shown in FIG. 9 and FIG. 10, the rest-back pad 53 is formed to extend more narrowly as approaching to the waist from the neck portion as well as to have one main surface 53a in contact with the user's back, having a curved surface at the waist portion set to a steeper slope than that at the neck portion.

The fixing belt 52 is a belt for fixing and supporting the end portion at the waist portion of the back rest 51 formed lengthily so the end portion as to be pushed as unmovable to the user. On the fractured clavicle fixing band, even though the back rest 1 is formed lengthily, since the position where the shoulder belts 2 are engaged with the back rest 1 is the same as that in the first embodiment, undesirably, the end portion at the waist portion of the back rest 51 is hardly affected by the fastening force by the shoulder belts 2 having function such as fixing and supporting as pushing the back rest 51 to the user's back in an unmovable manner. On the fractured clavicle fixing band, the back rest 51 is therefore subject to shift upon affected by the external force, so that it becomes difficult to support firmly the orderly reset region. Thus, to fix and support the end portion at the waist portion of the back rest 51 formed lengthily as pushed to the user in an unmovable manner, the fixing belt 52 is formed to the end portion at the waist portion of the back rest 51.

The fixing belt 52 comprises, as shown in FIG. 7 and FIG. 8 for example, two belt halves 52a, 52b formed of an ordinary fabric belt material stretched as passed from the user's back portion in left and right directions respectively, in which the belt halves 52a, 52b are coupled together in front of the user. Upon coupling the belt halves 52a, 52b, the back rest 51 is pulled and fastened as pushed to the user, thereby preventing the back rest 51 from shifting upon affected by the external force, so that the orderly reset region can be firmly supported.

Upon forming the back rest 51 lengthily, the thus structured fractured clavicle fixing band according to this embodiment can keep a wide contact surface in contact with the user so as to be able firmly support the orderly reset region more stably, in addition to the same effect as the above described first embodiment. Furthermore, even where formed lengthily, the back rest 51 is not shifted since firmly fixed as supported to the user with the fixing belt 52.

This invention is, needless to say, not limited to the structure of each embodiment as described above but be able to be deformed variously as needed within a range of the purpose of this invention.

Industrial Applicability

As described above in detail, the antagonistic action between the fastening force with the shoulder belt and the fastening force with the chest front belt occurs according to this invention, thereby causing the more stable fixing force, so that the action with the chest front belt provides stronger stability to such an effect as pulling the bone fragments posterosuperior, caused upon the function of the shoulder, such as redressing the clavicle in a backward direction and pressuring the clavicle in directions of external ends thereof. According to this invention, the clavicle region therefore stops completely swinging in backward and forward directions, thereby preventing effectively the orderly reset region from being dislocated. According to this invention, the orderly reset region is supported effectively as described above, thereby being able to suppress side effects such as, e.g., pain or stiffness in the shoulder due to excessive tension at the muscle in the neck, the shoulder, the back, etc., or the neural paralysis at the axilla or the pressure of the axillary artery due to pain in the joint or the pressure of the axillary nerve, conventionally caused due to the shoulder belt fastened excessively strongly.

The fractured clavicle fixing band according to this invention enables the user, even in a supine position, to keep his chest thrown out sufficiently upon equipment of the back-rest pad to the back rest, thereby being able to suppress the orderly reset region more effectively from being dislocated, so that any significant positional changes of the body in backward and forward directions between, e.g., the daytime and nighttime, can be solved.

1. A fractured clavicle fixing band comprising:
a back rest rendered in contact with a back of a user along a backbone when wore;
a pair of shoulder belts connected to an end portion of the back rest, stretched as respectively passed from shoulders via axillae when wore; and
a chest front belt for pulling and fastening, in front of a chest of the user, the pair of the shoulder belts in a direction to narrow a distance therebetween, wherein the front chest belt is made of a non-expandable material.

2. The fractured clavicle fixing band according to claim 1, further comprising a back-rest pad equipped to the back rest, wherein the back-rest pad has one main surface located at a side in contact with the back of the user forming a curved surface, and has a prescribed height enough to render the user threw out his chest.

3. The fractured clavicle fixing band according to claim 2, wherein the back-rest pad is detachably attached to the back rest.

4. The fractured clavicle fixing band according to claim 1, wherein the back rest has a length from a position slightly below a neck to an approximately center portion of the back of the user when wore, and wherein a fixing belt stretched as passed along a body of the user, for fixing and supporting the back rest, is formed to an end portion at the waist portion of the back rest.

5. A fractured clavicle fixing band comprising:
a back rest rendered in contact with a back of a user along a backbone when used;
a pair of shoulder belts connected an end portion of the back rest, stretched as passed from a shoulder to an axilla when used; and
a back-rest pad equipped to the back rest, wherein the back-rest pad has one main surface at a side in contact with the back of the user, set to a curved surface, and has a prescribed height enough to render the user threw out his chest.
